(19) United States
(12) Patent Application Publication
(10) Pub. No.: US 2007/0020657 A1
(43) Pub. Date: Jan. 25, 2007

(54) Methods for detecting circulating tumor cells

(76) Inventors: Stefan K.G. Grebe, Rochester, MN (US); Mark R. Pittelkow, Rochester, MN (US); Kevin C. Halling, Rochester, MN (US); Aleksandar Sekulic, Rochester, MN (US); Kendall W. Cradic, Rochester, MN (US); Ming Mai, Rochester, MN (US)

Correspondence Address: FISH & RICHARDSON P.C., PO BOX 1022, MINNEAPOLIS, MN, 55440-1022, US

(21) Appl. No.: 11/437,541

(22) Filed: May 19, 2006

Related U.S. Application Data
(60) Provisional application No. 60/682,987, filed on May 20, 2005.

(51) Int. Cl. C12Q 1/68; C12P 19/34

(52) U.S. Cl. 435/6; 435/91.2

(57) ABSTRACT

Nucleic acids and methods for amplifying and detecting mutant BRAF sequences are provided herein. In particular, nucleic acids and methods for amplifying and detecting BRAF sequences that encode the V600E BRAF mutant are provided herein.

### CROSS-REFERENCE TO RELATED APPLICATION

This application claims the benefit of priority from U.S. Provisional Application Ser. No. 60/682,987, filed May 20, 2005, the disclosure of which is incorporated herein by reference in its entirety.

### TECHNICAL FIELD

This invention relates to methods for detecting tumor cells in the circulation, and more particularly to methods for detecting tumor cells having a mutant BRAF gene.

### BACKGROUND

A percentage of human cancers, regardless of type, contain a somatic mutation in the BRAF gene. The mutation results in substitution of a valine (V) residue with a glutamic acid (E) residue. This change is particularly common in melanomas and papillary thyroid carcinomas, with 30-50% of tumors carrying the mutation. The BRAF mutation also occurs at a lesser frequency in many other human tumors, including colon tumors. Affected cells typically are heterozygous for the mutation, but can be homozygous.

### SUMMARY

The substitution of glutamic acid for valine at position 600 (formerly numbered as position 599) of the BRAF protein is referred to herein as V600E. The present document is based in part on the discovery that cancer metastasis can be detected by testing the blood (e.g., whole blood, serum, or plasma) of patients for the presence of cells that carry the V600E BRAF mutation. Since none of a patient's other body tissues carry this mutation, tumor spread to the blood stream, which may precede metastasis to other organs, can be an early indicator of cancer progression. The inventors have developed a technique that allows reliable detection of tumor cells (e.g., circulating tumor cells) carrying the V600E BRAF mutation in human biological samples. This method can include, for example, specific amplification and detection of only the mutated form of BRAF in DNA extracted from blood or tissue samples. Methods described herein also can include amplification across the region of the mutation followed by specific detection of only the mutant. These methods are applicable to all cancer patients whose original tumor, regardless of type, contains the V600E BRAF mutation.

In one aspect, this document features a method for determining whether a subject contains, in the peripheral circulation, a nucleic acid containing a mutant BRAF gene or a fragment thereof. The method can include: (a) providing nucleic acid from a peripheral blood sample obtained from the subject; (b) contacting the nucleic acid with at least a first primer under conditions that will result, if a BRAF gene or fragment thereof is present in the peripheral blood sample, in amplification of the BRAF gene or fragment thereof; and (c) determining whether the BRAF gene or fragment thereof contains a mutation as compared to a wild-type BRAF sequence. The subject can be a human. The subject can be diagnosed with cancer. The nucleic acid can be contained within cells (e.g., melanoma, papillary thyroid carcinoma, or colon cancer cells) in the peripheral circulation. The peripheral blood sample can be a serum sample or a plasma sample. The mutation can be an adenine substitution for thymine at nucleotide 1799 relative to the adenine in the BRAF translation initiation codon.

Step (b) of the method can include contacting the nucleic acid with at least a first primer under conditions that will result, if the mutant BRAF gene or fragment thereof is present in the peripheral blood sample, in specific amplification of the mutant BRAF gene or fragment thereof, giving a first amplified product, and step (c) can include detecting the presence or absence of the first amplified product, wherein the presence of the first amplified product indicates that the mutant BRAF gene or fragment thereof is present in the peripheral blood sample, and wherein the absence of the first amplified product indicates that the mutant BRAF gene or fragment thereof is not present in the peripheral blood sample. The first primer can be complementary to either strand of a wild-type BRAF nucleotide sequence, with the proviso that the nucleotide at the 3′ end of the first primer is not complementary to either strand of the wild-type BRAF nucleotide sequence. The first primer can have the sequence set forth in SEQ ID NO:3. The detecting can include gel electrophoresis, melting profile with an intercalating dye, hybridization with an internal probe, and/or real time PCR. The first primer can have a fluorescent label. The method can further include: (d) contacting the nucleic acid with at least a second primer under conditions that will result, if a non-mutant BRAF gene is present in the peripheral blood sample, in specific amplification of the non-mutant BRAF gene, giving a second amplified product; and (e) detecting the presence or absence of the second amplified product. The first primer can have the nucleotide sequence set forth in SEQ ID NO:3, and the second primer can have the nucleotide sequence set forth in SEQ ID NO:5. The method can further include comparing the amounts of the first amplified product and the second amplified product. When the nucleic acid is contained within cells in the peripheral circulation, the relative levels of the first and second amplified products can indicate the fraction of cells having the mutant BRAF gene in the peripheral blood sample. The method can further include, after step (a) and prior to step (b), contacting the nucleic acid with one or more degenerate primers under conditions that will result in universal amplification of the nucleic acid.

In another aspect, this document features a method for detecting residual disease in tissue of a subject diagnosed with cancer. The method can include: (a) providing nucleic acid from a tissue sample obtained from the subject; (b) contacting the nucleic acid with at least a first primer under conditions that will result, if a BRAF gene is present in cells of the tissue sample, in specific amplification of the BRAF gene, giving a first amplified product; and (c) determining whether the BRAF gene or fragment thereof contains a mutation compared to a wild-type BRAF sequence, wherein the presence of the mutation indicates that the tissue sample contains residual disease, and wherein the absence of the mutation indicates that the tissue sample does not contain residual disease. The subject can be a human. The cancer can be melanoma, papillary thyroid carcinoma, or colon cancer. The mutation can be a thymine to adenine substitution at nucleotide 1799 relative to the adenine in the translation initiation codon.

Step (b) can include contacting the nucleic acid with at least a first primer under conditions that will result, if a mutant BRAF gene is present in cells of the tissue sample, in specific amplification of the mutant BRAF gene, giving a first amplified product, and wherein the step (c) comprises detecting the presence or absence of the first amplified product, wherein the presence of the first amplified product indicates that the tissue sample contains residual disease, and wherein the absence of the first amplified product indicates that the tissue sample does not contain residual disease. The first primer can be complementary to either strand of a wild-type BRAF nucleotide sequence, with the proviso that the nucleotide at the 3′ end of the first primer is not complementary to either strand of the wild-type BRAF nucleotide sequence. The first primer can have the sequence set forth in SEQ ID NO:3. The first primer can have a fluorescent label. The detecting can include gel electrophoresis, melting profile with an intercalating dye, hybridization with an internal probe, and/or real time PCR.

The method can further include: (d) contacting the nucleic acid with at least a second primer under conditions that will result, if a non-mutant BRAF gene is present in cells of the tissue sample, in specific amplification of the non-mutant BRAF gene, giving a second amplified product; and (e) detecting the presence or absence of the second amplified product. The method can further include comparing the amounts of the first amplified product and the second amplified product, wherein the relative levels of the first and second amplified products indicates the fraction of cells having the mutant BRAF gene in the tissue sample. The first primer can have the nucleotide sequence set forth in SEQ ID NO:3, and the second primer can have the nucleotide sequence set forth in SEQ ID NO:5. The method can further include, after step (a) and prior to step (b), contacting the nucleic acid with one or more degenerate primers that will result in universal amplification of the nucleic acid.

In another aspect, this document features an isolated nucleic acid having the nucleotide sequence set forth in SEQ ID NO:3. In invention also features an isolated nucleic acid having the nucleotide sequence set forth in SEQ ID NO:4.

In yet another aspect, this document features an article of manufacture including an isolated nucleic acid having the nucleotide sequence set forth in SEQ ID NO:3, and an isolated nucleic acid having the nucleotide sequence set forth in SEQ ID NO:4. The article of manufacture can further include an isolated nucleic acid having the nucleotide sequence set forth in SEQ ID NO:5.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a reference BRAF nucleotide sequence (SEQ ID NO:1). The translation initiation codon and the stop codon are bold and underlined. The nucleotide at position 1799 relative to the adenine in the translation initiation start codon is circled. FIG. 1B is the reference BRAF amino acid sequence (SEQ ID NO:2). The valine at position 600 is circled.

FIG. 2 is a graph showing levels of wild-type and mutant BRAF nucleic acids as detected using Sybr Green.

FIG. 3 is a graph showing levels of mutant and wild-type BRAF nucleic acids as detected using hybridization probes.

FIG. 4 is a graph showing a dilution series of melanoma cells carrying one copy of the BRAF V600E mutant (heterozygotes) in blood samples from healthy volunteers. Detection was accomplished with Sybr Green. The cell-dilution factor was calculated based on white blood cell counts in the blood samples.

FIG. 5 is a graph showing a dilution series of melanoma cells carrying one copy of the BRAF V600E mutant (heterozygotes) in blood samples from healthy volunteers. Detection was accomplished with labeled primers and hybridization probes (see Table 1). The cell-dilution factor was calculated based on white blood cell counts in the blood samples.

FIG. 6 is a graph showing wild-type and mutant BRAF nucleic acids detected in a patient sample and a control sample using Sybr Green.

FIG. 7 is a graph showing amplification curves for wild-type BRAF in serum from normal volunteers. Curves are from regular template as well as whole-gene amplified (WGA) template, as indicated.

FIG. 8 is a graph showing amplification curves for wild-type BRAF in plasma from normal volunteers. Curves are from regular template as well as WGA template, as indicated.

FIG. 9 is a graph showing amplification curves for mutant BRAF in plasma samples from stage IV melanoma patients.

FIG. 10 is a graph showing amplification curves for wild-type BRAF in plasma samples from stage IV melanoma patients.

### DETAILED DESCRIPTION

Circulating cancer cells can be markers of metastatic disease, or can indicate increased risk of future development of metastatic disease. Detection of tumor cells in blood by conventional immunochemical or cytogenetic methods has proven difficult, mainly due to the limited sensitivity of these techniques. In contrast, extremely sensitive molecular identification of a variety of cell types has become possible with the development of PCR (Johnson et al. (1995) Br. J. Cancer 72:268-276; and Lacroix and Doeberitz (2001) Sem. Surg. Oncol. 20:252-264). Most of the PCR-based assays that have been developed detect the presence of tumor cells in blood based on reverse transcription and subsequent amplification of tumor-associated mRNAs from whole blood. If tumor cells are present, PCR will amplify these transcripts, whereas if tumor cells are absent the reaction will fail. While the sensitivity of these mRNA-based methods generally is good, issues such as RNA degradation and low efficiency of the reverse transcriptase reaction can severely limit the practical usability of these types of assay. In addition, because the amount of tumor-specific mRNA produced can vary widely depending on the metabolic state of the circulating cells, the assays are plagued with poor reproducibility.

Methods based on molecular detection of genomic tumor-specific DNA can overcome most of the limitations of RNA-based assays, since genomic DNA is exceptionally stable and there is no need for reverse transcription. However, because all cells, whether malignant or benign, share the same DNA, these assays only can be used when a tumor has acquired a specific DNA alteration that distinguishes it from non-tumorous cells.

If these conditions are fulfilled, any cell type that carries target DNA can be detected on a background of normal cells. In particular, high sensitivity of PCR-based assays, which achieve exponential amplification of the mutated DNA prior to detection, can allow detection of even a single tumor cell among thousands of normal cells in peripheral blood. The problem with this approach is that the types of genetic alterations observed in tumors tend to vary from one tumor to the next, and from one patient to the next. Thus, in most cases individualized tumor genome profiling would be a prerequisite for detection of circulating tumor cells having tumor-specific mutations. This can be costly, time- and labor-intensive, and may not be sufficiently reliable.

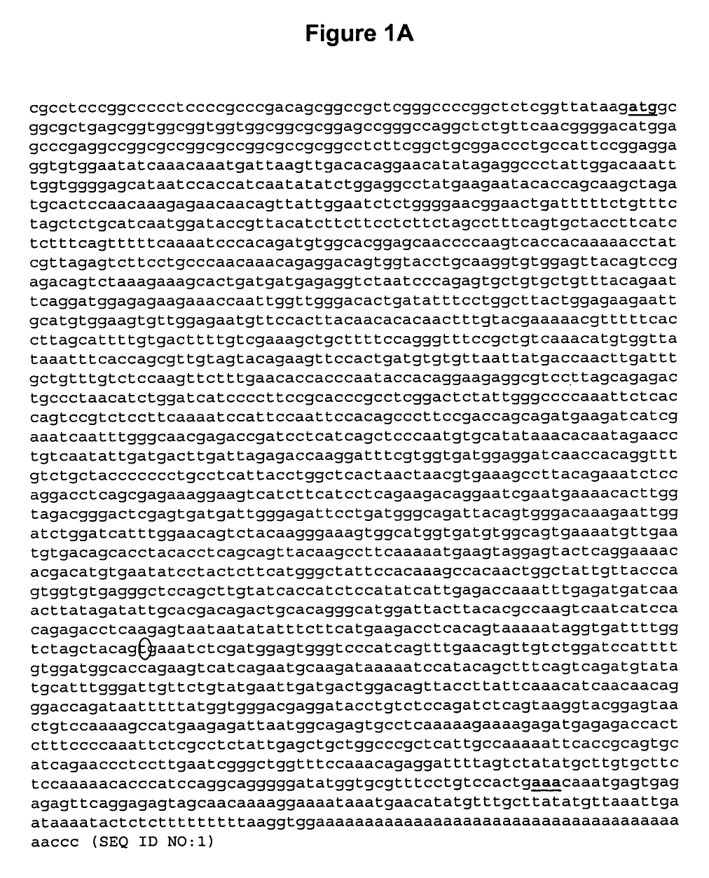

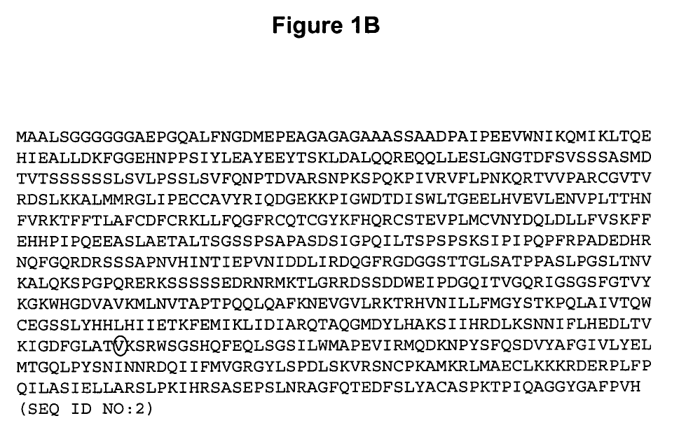

The inventors have discovered, however, that a particular tumor-specific mutation lends itself well to DNA-based detection of tumor cells, and particularly tumor cells that have entered the peripheral circulation. In many human tumors, the BRAF gene carries a single point mutation in exon 15. This mutation is a thymine to adenine substitution at nucleotide 1799 relative to the adenine in the translation initiation codon, which is considered to be nucleotide 1. A reference BRAF sequence shown in FIG. 1A (SEQ ID NO:1). The mutation at nucleotide 1799 results in substitution of glutamic acid for valine at amino acid 600 (Davies et al. (2002) Nature 417:949-954; designated as amino acid 599 in the Davies et al. reference), and is referred to herein as V600E. A reference BRAF amino acid sequence is shown in FIG. 1B (SEQ ID NO:2). The V600E mutation causes constitutive activation of BRAF, and has been detected in a variety of cancers including 67% of melanomas and 36% of papillary thyroid carcinomas (PTC) (Davies et al., supra; and Kimura et al. (2003) Cancer Res. 63:1454-1457). Thus, the V600E BRAF mutation is the most common genetic mutation found in melanoma and PTC. Many other human tumor types (e.g., colon cancer) also carry this mutation, albeit at a lower frequency of about 5-15%. Because metastatic spread of cancers often occurs hematogenously, detection of nucleic acids containing the V600E BRAF mutation in the peripheral circulation can serve as a means of identifying disease recurrence in patients whose primary tumor carries the mutation.

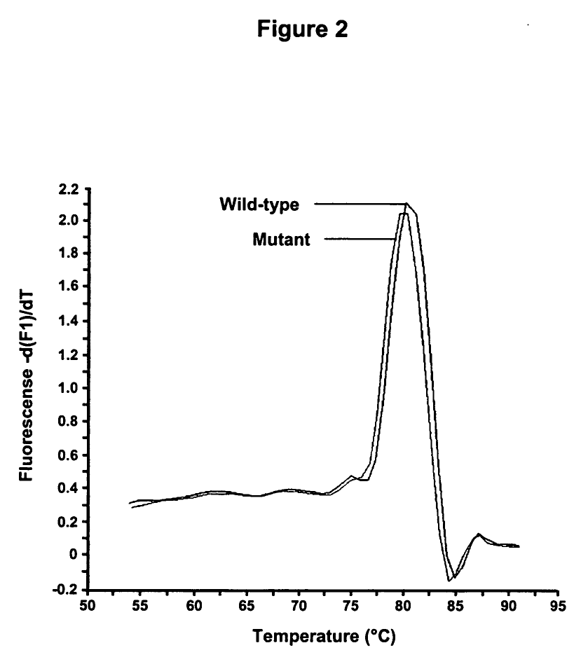

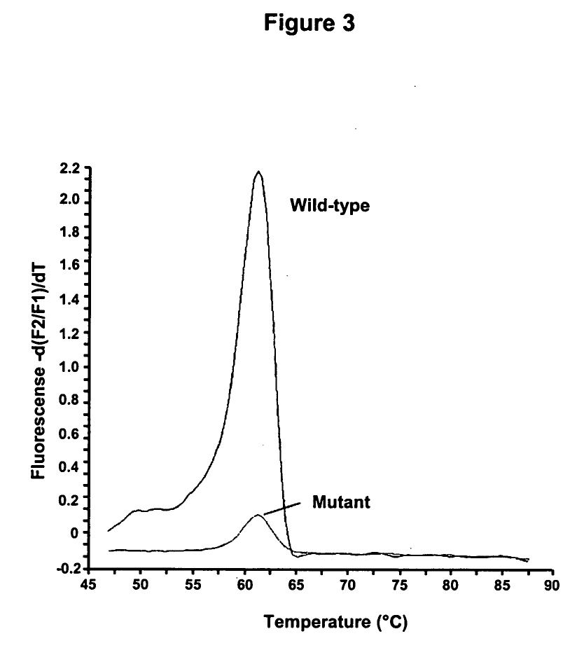

The inventors have developed an assay for nucleic acids in peripheral blood that carry the V600E BRAF mutation. The nucleic acids can be contained within cells or can be free within the peripheral blood. This assay involves PCR and a sequence-specific priming strategy using the primers shown in Table 1. A primer set that is specific to the mutant sequence and another that is specific to the wild-type sequence can be used to selectively amplify either form of the BRAF gene for subsequent detection. A variety of methods can be used to detect an amplified product, including gel electrophoresis with fluorescent dyes, melting profiles with intercalating dyes (FIG. 2), and hybridization with internal probes (FIG. 3). The inventors have successfully used all three of these techniques (see the Examples herein). In each case, mutant BRAF can be detected reliably. In addition, by comparing the detection signal intensity of reactions using the wild-type and mutant primers with a single patient sample, an estimate of the fraction of cells in a blood sample that carry the mutation can be made.

A major challenge for such an assay is specificity, i.e., lack of false positive results. The methods disclosed herein are extremely specific. As described in the Examples, no positives were detected in samples from 90 healthy individuals. Further, no positives were detected in samples from 22 thyroid cancer patients without distant metastatic disease.

TABLE 1

Primers for specific amplification of
wild-type BRAF and V600E BRAF mutant

| | | SEQ |
|---|---|---|
| | | ID |
| Primer | Sequence | NO: |
| BRAF mutant | GATTTTGGTCATGCTACAGA | 3 |
| forward | | |
| BRAF reverse | CTTTCTAGTAACTCAGCAGC | 4 |
| BRAF wild-type | GATTTTGGTCATGCTACAGT | 5 |
| forward | | |
| BRAF wild-type 640 | GATTTTGGTCATGC*T*ACAGT | 5 |
| BRAF WT flr | CACTCCATCGCGATTTCACTG-flr | 6 |
| BRAF mut 640 | GATTTTGGTCATGC*T*ACAGA | 3 |
| BRAF mut flr | CACTCCATCGAGATTTCTCTG-flr | 7 |

*indicates the position of internal LC640 dye
-flr indicates a fluorescein label

1. Nucleic Acids

This document features isolated nucleic acids that can include a BRAF nucleic acid sequence. In some embodiments, the BRAF nucleic acid sequence can include a nucleotide sequence variant at nucleotide 1799 relative to the adenine in the translation initiation codon of the reference BRAF sequence shown in FIG. 1A (SEQ ID NO:1; also available in GenBank® under accession no. M95712), as well as nucleotides flanking position 1799. As used herein, “isolated nucleic acid” refers to a nucleic acid that is separated from other nucleic acid molecules that are present in a mammalian genome, including nucleic acids that normally flank one or both sides of the nucleic acid in a mammalian genome (e.g., nucleic acids that encode non-BRAF proteins). The term “isolated” as used herein with respect to nucleic acids also includes any non-naturally-occurring nucleic acid sequence since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

The nucleic acid molecules provided herein can be between about 8 and about 50 nucleotides in length. For example, a nucleic acid can be 8, 9, 10, 11, 12, 13, 14, 15, 16, 17, 18, 19, 20, 21, 22, 23, 24, 25, 26, 27, 28, 29, 30, 32, 34, 36, 38, 40, 45, or 50 nucleotides in length. Alternatively, the nucleic acid molecules provided herein can be greater than 50 nucleotides in length (e.g., 55, 60, 65, 70, 75, 80, 85, 90, 95, 100, or more than 100 nucleotides in length). Nucleic acid molecules can be in a sense or antisense orientation, can be complementary to a BRAF reference sequence (e.g., the sequence shown in GenBank® accession no. M95712.2), and can be DNA, RNA, or nucleic acid analogs. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine or 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, Summerton and Weller, Antisense Nucleic Acid Drug Dev. (1997) 7(3):187-195; and Hyrup et al. (1996) Bioorgan. Med. Chem. 4(1):5-23. In addition, the deoxyphosphate backbone can be replaced with, for example, a phosphorothioate or phosphorodithioate backbone, a phosphoroamidite, or an alkyl phosphotriester backbone.

The isolated nucleic acid molecules provided herein can be produced using standard techniques including, without limitation, chemical synthesis. Examples of nucleic acid molecules provided herein are shown in Table 1, and have the nucleotide sequences set forth in SEQ ID NOS:3, 4, and 5.

2. Methods

The invention provides methods for determining whether a subject (e.g., a mammal such as a human) contains cells having a mutant BRAF gene. The methods provided herein can be useful to determine whether a subject may have metastasizing cancer, or to detect residual (e.g., minimal residual) disease in a subject. The subject can be diagnosed with cancer, or can be a healthy individual. The cells can be any type of cells, including, without limitation, cancer cells such as melanoma cells, PTC cells, colon cancer cells, glioma cells, sarcoma cells, rhabdomyosarcoma cells, hepatocellular carcinoma cells, breast cancer cells, prostate cancer cells, or squamous cell carcinoma cells).

The methods provided herein can include obtaining a biological sample from a subject. As used herein, a “biological sample” is a sample that contains cells or cellular material. Non-limiting examples of biological samples include urine, blood, plasma, serum, cerebrospinal fluid, pleural fluid, sputum, peritoneal fluid, bladder washings, secretions (e.g., breast secretion), oral washings, tissue samples, tumor samples, touch preps, or fine-needle aspirates. A biological sample can be obtained using any suitable method. For example, a blood sample (e.g., a peripheral blood sample) can be obtained from a subject using conventional phlebotomy procedures. Similarly, plasma and serum can be obtained from a blood sample using standard methods.

Methods of the invention can further include isolating nucleic acids from the biological sample. Nucleic acids can be isolated using any method. For example, DNA from a peripheral blood sample can be isolated using a DNeasy DNA isolation kit, a QIAamp DNA blood kit, or a PAXgene blood DNA kit from Qiagen Inc. (Valencia, Calif.). DNA from other tissue samples also can be obtained using a DNeasy DNA isolation kit. Any other DNA extraction and purification technique also can be used, including liquid-liquid and solid-phase techniques ranging from phenol-chloroform extraction to automated magnetic bead nucleic acid capture systems.

Once nucleic acid has been obtained, it can be contacted with at least one oligonucleotide (e.g., a primer) that can result in specific amplification of a mutant BRAF gene (e.g., a BRAF sequence having an adenine in place of a thymine at position 1799) if the mutant BRAF gene is present in the biological sample. As used herein, the term “specific amplification” means that under particular conditions, an oligonucleotide can interact with and prime amplification of a particular nucleotide sequence (e.g., a BRAF sequence containing a thymine to adenine substitution at position 1799), without priming detectable amplification of other nucleotide sequences (e.g., a wild-type BRA F sequence containing position 1799) potentially present in the biological sample. The nucleic acid also can be contacted with a second oligonucleotide (e.g., a reverse primer) that hybridizes to either a mutant or a wild-type BRAF gene. The nucleic acid sample and the oligonucleotides can be subjected to conditions that will result in specific amplification of a portion of the mutant BRAF gene if the mutant BRAF gene is present in the biological sample. For example, the first oligonucleotide can have the sequence set forth in SEQ ID NO:3, and the second oligonucleotide can have the sequence set forth in SEQ ID NO:4. A primer can be labeled internally with a dye (e.g., LC640 dye). Further, a probe can be labeled with fluorescein, and can be designed to bind to the nascent strand opposite from the LC640 dye, allowing for FRET transfer across the helix.

The conditions used for amplification can include, for example, reaction mixture containing 1×PCR buffer (ABI), 1.5 mM MgCl₂, 1 mg/ml BSA, 200 μM dNTPs, 7.5 units polymerase (e.g., Amplitaq Gold®; Applied Biosystems, Foster City, Calif.), 600 μM reverse primer, 400 μM forward primer labeled with LC640, 200 μM fluorescein probe, and 0.5 μg genomic DNA. Each analysis may require two reactions, one for amplification of the wild-type sequence and the other for the mutant. Reactions can be carried out in 20 μl LightCycler capillaries. Amplification can include an initial activation step at 95° C. for 10 minutes, followed by 50 to 60 cycles of annealing at 58° C. for 1 minute (transition rate of 3° C./second) and melting at 95° C. for 15 seconds. Fluorescence measurements can be made at the end of each annealing step. Melting curve data can be collected between 45° C. and 85° C.

Once the amplification reactions are completed, the presence or absence of an amplified product can be detected using any suitable method. Such methods include, without limitation, those known in the art, such as gel electrophoresis with or without a fluorescent dye (depending on whether the product was amplified with a dye-labeled primer), a melting profile with an intercalating dye, and hybridization with an internal probe. Alternatively, the amplification and detection steps can be combined in a real time PCR assay. Detection of an amplified product indicates that cells containing a mutant BRAF gene were present in the biological sample, while the absence of an amplified product indicates that cells containing a mutant BRAF gene were not present in the biological sample. The absence of such cells can further indicate that cancer in the subject has not metastasized.

The methods provided herein also can include contacting the nucleic acid sample with a third oligonucleotide (e.g., a primer having the sequence set forth in SEQ ID NO:5) that can result in specific amplification of a wild-type BRAF gene without detectable amplification of a mutant BRAF gene having an adenine substitution for thymine at nucleotide 1799. These methods can further include subjecting the nucleic acid and the oligonucleotides to conditions that will result in specific amplification of a wild-type BRAF sequence if a wild-type BRAF gene is present in the biological sample. The presence or absence of an amplified product containing a wild-type BRAF sequence can be detected using any suitable method, including those disclosed above. Methods that include using oligonucleotides for amplification of both mutant and wild-type BRAF sequences also can include quantifying and comparing the amounts of amplified product for each sequence. The relative levels of mutant and wild-type products can indicate the fraction of cells in the biological sample that contain a mutant BRAF gene. Lower fractions of cells containing the mutant BRAF sequence can indicate lower levels of metastasis, while higher fractions of cells containing the mutant sequence can indicate higher levels, or more severe, metastasis.

Alternatively, the methods provided herein can include amplification of a BRAF nucleic acid using primers that flank position 1799 relative to the adenine in the translation initiation codon. The amplified product then can be detected using, for example, a probe specific for the mutant sequence or a probe specific for the wild-type sequence. Alternatively, the amplicon can be sequenced to determine whether the BRAF nucleic acid encodes a polypeptide containing the V600E mutation.

In some embodiments, the methods disclosed herein can further include a first, universal amplification step. Such methods can include contacting nucleic acids obtained from a biological sample with, for example, a cocktail of degenerate primers, and using standard PCR procedures for an overall amplification of the DNA. This preliminary amplification can be followed by specific amplification and detection of products, as described herein.

3. Articles of Manufacture

One or more isolated nucleic acid molecules provided herein can be combined with packaging material and sold as a kit for detecting cells that contain a mutant BRAF gene. Components and methods for producing articles of manufactures are well known. The articles of manufacture typically contain an isolated nucleic acid molecule having the sequence set forth in SEQ ID NO:3. An article of manufacture further may contain an isolated nucleic acid molecule having the sequence set forth in SEQ ID NO:4 and/or SEQ ID NO:5. In addition, the articles of manufacture may further include buffers and other reagents for amplifying and/or detecting nucleic acid sequences. Instructions describing how the various nucleic acid molecules are effective for amplifying mutant and wild-type BRAF sequences also may be included in such kits.

The invention will be further described in the following examples, which do not limit the scope of the invention described in the claims.

### EXAMPLES

### Example 1

### Detection of the V600E BRAF Mutation in PTC Tumor and Blood Samples

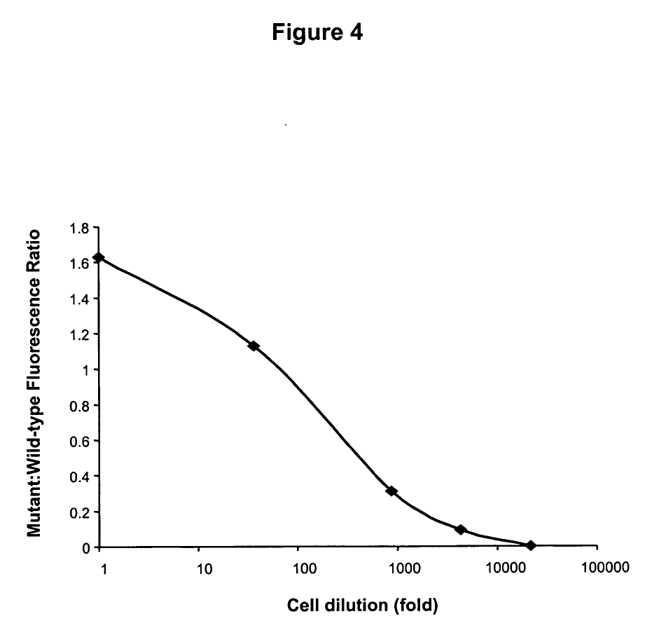

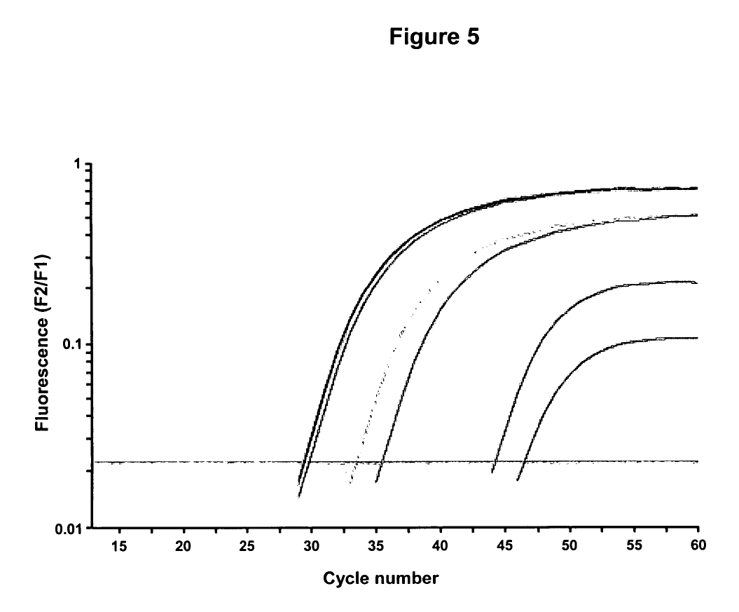

Using primers shown in Table 1 and the amplification conditions disclosed above, DNA from 19 archival PTC tumor samples was evaluated to determine the frequency of the V600E BRAF mutation in the patient population. Results were confirmed by dye-terminator sequencing. The nucleic acid substitution associated with the V600E BRAF mutation was detected in 47% of the PTC specimens tested. Serial dilutions of DNA from a V600E mutation-positive PTC specimen were prepared in normal genomic DNA to determine the ability of real-time PCR to detect various concentrations of mutant DNA in a background of normal genomic DNA. Mutant DNA was identified at a dilution of greater than 1:100,000. A cell dilution series was then prepared using the human melanoma cell line A375, which is heterozygous for the V600E BRAF mutation. A375 cells were used to spike blood samples from a healthy volunteer, followed by DNA extraction and real-time PCR. The ratios of real-time PCR crossing points of BRAF wild-type versus BRAF mutant were used to calculate that, at a minimum, one A375 tumor cell among about 40,000 normal leukocytes was detected using Sybr Green (FIG. 4). A similar experiment with higher DNA input, LC640-labeled primers, and fluorescein-labeled probes as shown in Table 1 yielded a detection sensitivity of 1:100,000 (FIG. 5). These results closely reflected the theoretical optimum sensitivity for the reaction conditions used.

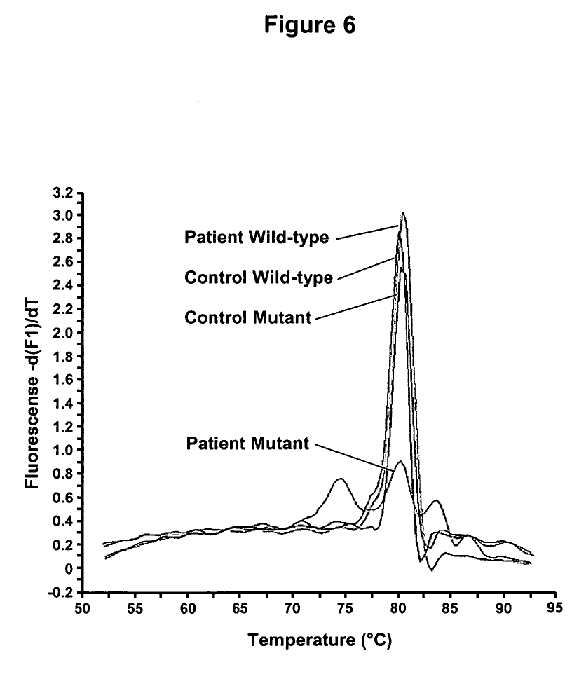

To assess the specificity of the assay, blood from 90 normal, healthy control subjects was tested. None of these samples showed evidence of circulating cells containing the V600E BRAF mutation. In addition, DNA extracted from blood samples of eight patients with a history of PTC was tested for the presence of the V600E BRAF mutation. The mutation was detected in at least one patient (FIG. 6). In a separate experiment, blood from 22 patients with PTC who were either free of disease or had only local nodal recurrence (i.e., no blood-borne tumor spread) was tested for the presence of the V600E mutation. All were negative for the mutation.

These data indicate that the V600E BRAF mutation, the most common somatic tumor-genetic change in PTC and melanoma, can be detected in peripheral blood samples using real-time PCR. The method is extremely specific. Thus, hematogenous spread of such cancers can be detected with high analytical sensitivity and excellent clinical specificity. This assay is applicable to any other human cancer where the primary tumor carries the V600E BRAF mutation.

### Example 2

### Detection of the V600E BRAF Mutation in Plasma and Serum

Since cells derived from solid tumors are not well adapted to the unique rigors of the cardiovascular system, they might be lysed, resulting in free tumor DNA in the circulation. Thus, plasma may be enriched in tumor-derived DNA. By detecting tumor-specific DNA variations in serum or plasma, subclinical primary or metastatic disease might be diagnosed with greater accuracy than in whole blood. In addition, use of serum or plasma allows utilization of larger range of sample-types that might be received routinely in a laboratory, as well as use of archived refrigerated or frozen test samples.

The techniques for BRAF detection in blood therefore were modified to additionally allow for detection of the V600E BRAF mutant and wild-type alleles in serum and plasma samples. DNA was extracted from plasma and serum using a modified Puregene™ (Gentra Systems, Inc., Minneapolis, Minn.) protocol. The solutions used were all components of the Puregene™ DNA purification kit, with the exception of proteinase K, isopropyl alcohol, ethanol, and glycogen. For each extraction, 1 ml of serum or plasma was diluted with 5 ml of cell lysis solution. After complete mixing, 18 U of proteinase K were added, and the samples were incubated at 55° C. for two hours. An additional 18 U of proteinase K were then added, and samples were incubated at 55° C. overnight. Samples were allowed to cool to room temperature, and 2 ml of protein precipitation solution were added to remove any remaining protein. Samples were then placed on ice for 1 hour, followed by centrifugation at 8,000 g for 70 minutes in a refrigerated centrifuge. Supernatants were poured into new tubes, and 6 ml of isopropyl alcohol and 10 μl of molecular biology grade glycogen were added to each sample. After gentle mixing, extractions were incubated for 5 minutes at room temperature. Samples were again centrifuged for 35 minutes at 8,000 g, and the resulting supernatants were removed. Six ml of 70% ethanol were added to each tube and, after gentle mixing, samples were again centrifuged at 8,000 g for 15 minutes. The supernatants were removed and the tubes were allowed to dry on absorbent paper. Hydration solution (100 μl) was added to each tube, and DNA was allowed to re-hydrate for 1 hour in a 60° C. incubated shaker and then overnight at room temperature.

DNA extracted from both serum and plasma was used as template material for the BRAF V600E assay developed for the LightCycler (see above).

To improve efficiency for the more fragmented DNA that is usually found in serum or plasma, one of the following alternative reverse primers, which result in shorter amplicons, can also be used: 5′-CAATTCTTACCATCCACAAAATG-3′ (SEQ ID NO:8), 5′-CCATCCACAAAATGGATCCAGAC-3′ (SEQ ID NO:9), 5′-CAAAATGGATCCAGACAACTGTTCAAAC-3′ (SEQ ID NO:10), or 5′-GGATCCAGACAACTGTTCAAAC-3′ (SEQ ID NO:11).

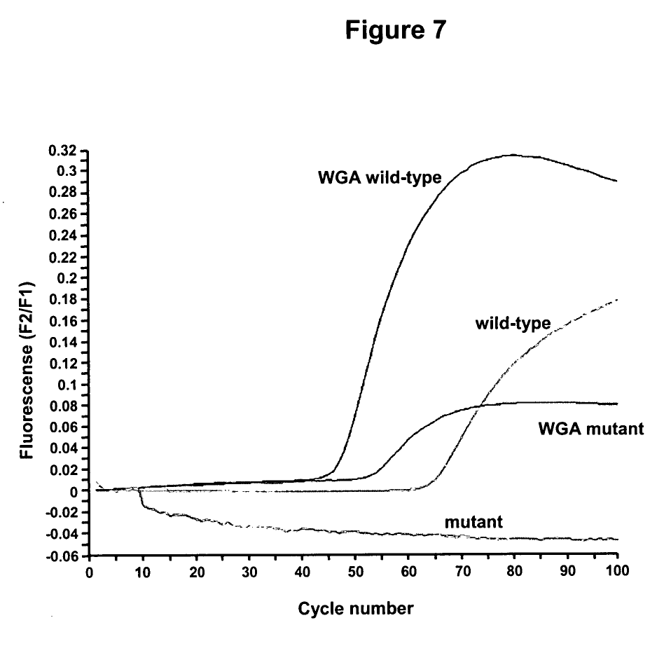

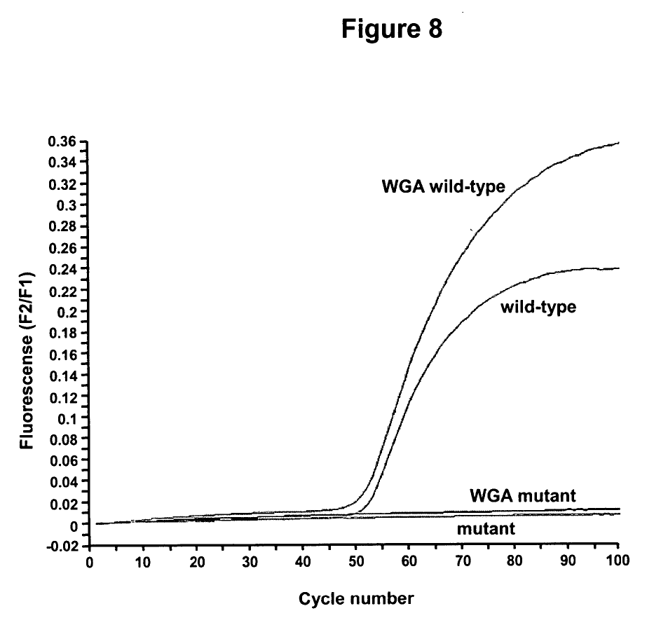

FIGS. 7 and 8 show amplification curves for wild-type and mutant BRAF in serum and plasma, respectively, from normal volunteers. While amplification was achieved, DNA copy numbers appeared low. To increase the overall sensitivity, whole gene amplification (WGA) of the extracted DNA was performed before allele specific PCR of some samples, as indicated in the figures. A REPLI-g® multiple displacement amplification (MDA) kit from Qiagen, Inc. was used in these experiments, according to the manufacturer's protocol. Following universal amplification, an aliquot of the reaction was used in allele specific BRAF wild type and BRAF mutant PCR as described above.

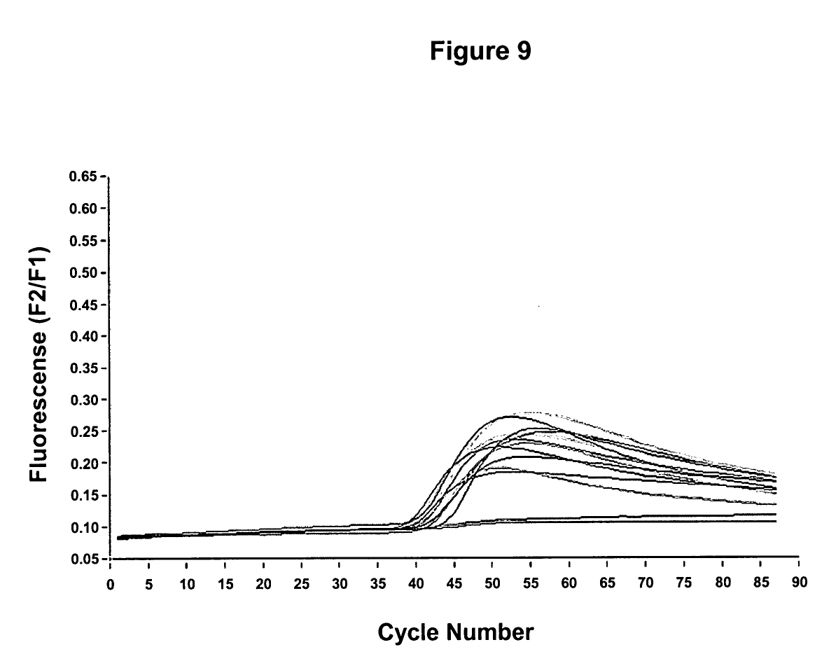

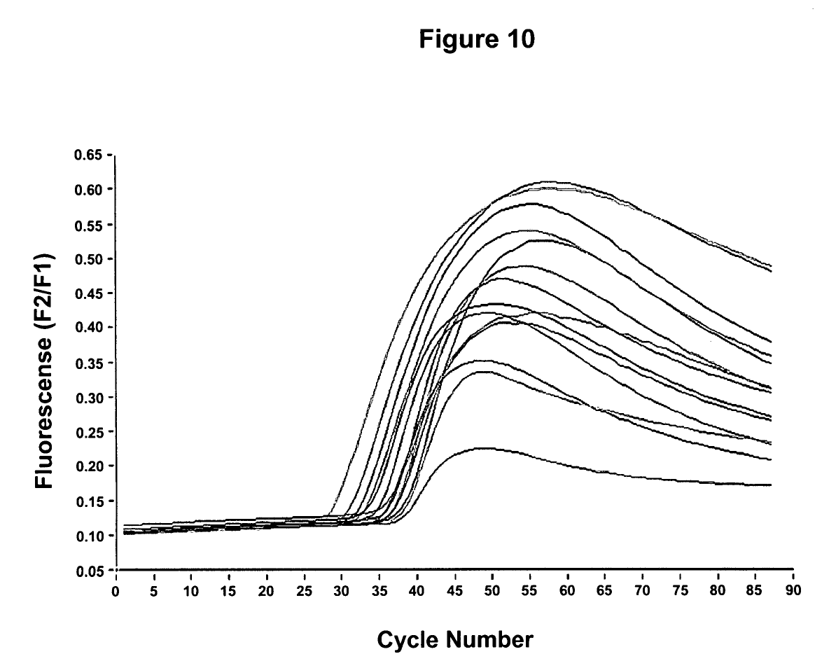

FIGS. 9 and 10 show amplification curves for mutant and wild-type BRAF in plasma samples from stage IV melanoma patients. These samples were assayed for wild-type and mutant sequences. Eleven of the 14 samples (79%) contained detectable levels of mutant BRAF DNA (FIG. 10), while all 14 samples (100%) contained detectable levels of the wild-type BRAF DNA. Taken together, these experiments demonstrated that plasma and serum are useful sources of tumor DNA for detection of the V600E BRAF mutation.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining whether a subject contains, in the peripheral circulation, a nucleic acid comprising a mutant BRAF gene or a fragment thereof, said method comprising:
(a) providing nucleic acid from a peripheral blood sample obtained from said subject;
(b) contacting said nucleic acid with at least a first primer under conditions that will result, if a BRAF gene or fragment thereof is present in said peripheral blood sample, in amplification of said BRAF gene or fragment thereof; and
(c) determining whether said BRAF gene or fragment thereof contains a mutation as compared to a wild-type BRAF sequence.

2. The method of claim 1, wherein said subject is a human.

3. The method of claim 1, wherein said subject is diagnosed with cancer.

4. The method of claim 1, wherein said nucleic acid is contained within cells in the peripheral circulation.

5. The method of claim 4, wherein said cells are melanoma, papillary thyroid carcinoma, or colon cancer cells.

6. The method of claim 1, wherein said peripheral blood sample is a serum sample.

7. The method of claim 1, wherein said peripheral blood sample is a plasma sample.

8. The method of claim 1, wherein said mutation is an adenine substitution for thymine at nucleotide 1799 relative to the adenine in the BRAF translation initiation codon.

9. The method of claim 1, wherein said step (b) comprises contacting said nucleic acid with at least a first primer under conditions that will result, if said mutant BRAF gene or fragment thereof is present in said peripheral blood sample, in specific amplification of said mutant BRAF gene or fragment thereof, giving a first amplified product, and wherein said step (c) comprises detecting the presence or absence of said first amplified product, wherein the presence of said first amplified product indicates that said mutant BRAF gene or fragment thereof is present in said peripheral blood sample, and wherein the absence of said first amplified product indicates that said mutant BRAF gene or fragment thereof is not present in said peripheral blood sample.

10. The method of claim 9, wherein said first primer is complementary to either strand of a wild-type BRAF nucleotide sequence, with the proviso that the nucleotide at the 3′ end of said first primer is not complementary to either strand of the wild-type BRAF nucleotide sequence.

11. The method of claim 9, wherein said first primer has the sequence set forth in SEQ ID NO:3.

12. The method of claim 9, wherein said detecting comprises gel electrophoresis, melting profile with an intercalating dye, hybridization with an internal probe, and/or real time PCR.

13. The method of claim 9, wherein said first primer comprises a fluorescent label.

14. The method of claim 9, further comprising
(d) contacting said nucleic acid with at least a second primer under conditions that will result, if a non-mutant BRAF gene is present in said peripheral blood sample, in specific amplification of said non-mutant BRAF gene, giving a second amplified product; and
(e) detecting the presence or absence of said second amplified product.

15. The method of claim 14, further comprising comparing the amounts of said first amplified product and said second amplified product.

16. The method of claim 15, wherein said nucleic acid is contained within cells in the peripheral circulation, and wherein the relative levels of said first and second amplified products indicates the fraction of cells having said mutant BRAF gene in said peripheral blood sample.

17. The method of claim 14, wherein said first primer has the nucleotide sequence set forth in SEQ ID NO:3, and wherein said second primer has the nucleotide sequence set forth in SEQ ID NO:5.

18. The method of claim 9, further comprising, after step (a) and prior to step (b), contacting said nucleic acid with one or more degenerate primers under conditions that will result in universal amplification of said nucleic acid.

19. An isolated nucleic acid having the nucleotide sequence set forth in SEQ ID NO:3.

20. An isolated nucleic acid having the nucleotide sequence set forth in SEQ ID NO:4.

21. An article of manufacture comprising an isolated nucleic acid having the nucleotide sequence set forth in SEQ ID NO:3, and an isolated nucleic acid having the nucleotide sequence set forth in SEQ ID NO:4.

22. The article of manufacture of claim 21, further comprising an isolated nucleic acid having the nucleotide sequence set forth in SEQ ID NO:5.


SEQUENCE LISTING (11 sequences)

SEQ ID NO: 1 - DNA, 2513 bp, Homo sapien
cgcctcccggccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttgggattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtggaaaaaaaaaaaaaaaaaaaaaaaaaaaaaaaaaaaaaaaccc

SEQ ID NO: 2 - protein, 766 aa, Homo sapien [sequence omitted]

SEQ ID NO: 3 - DNA, 20 bp, Artificial Sequence
  Primer
gattttggtcatgctacaga

SEQ ID NO: 4 - DNA, 20 bp, Artificial Sequence
  Primer
ctttctagtaactcagcagc

SEQ ID NO: 5 - DNA, 20 bp, Artificial Sequence
  Primer
gattttggtcatgctacagt

SEQ ID NO: 6 - DNA, 21 bp, Artificial Sequence
  Primer
cactccatcgcgatttcactg

SEQ ID NO: 7 - DNA, 21 bp, Artificial Sequence
  Primer
cactccatcgagatttctctg

SEQ ID NO: 8 - DNA, 23 bp, Artificial Sequence
  Primer
caattcttaccatccacaaaatg

SEQ ID NO: 9 - DNA, 23 bp, Artificial Sequence
  Primer
ccatccacaaaatggatccagac

SEQ ID NO: 10 - DNA, 28 bp, Artificial Sequence
  Primer
caaaatggatccagacaactgttcaaac

SEQ ID NO: 11 - DNA, 22 bp, Artificial Sequence
  Primer
ggatccagacaactgttcaaac